(19) United States
(12) Patent Application Publication
(10) Pub. No.: US 2013/0306081 A1
(43) Pub. Date: Nov. 21, 2013

(54) Neonatal Medical Apparatus and Methods

(71) Applicants: Srisatish Devapatla, Ithaca, NY (US); Madhavi D. Reddy, Ithaca, NY (US)

(72) Inventors: Srisatish Devapatla, Ithaca, NY (US); Madhavi D. Reddy, Ithaca, NY (US)

(21) Appl. No.: 13/473,998

(22) Filed: May 17, 2012

(51) Int. Cl. A61F 5/37; A61M 16/04

(52) U.S. Cl. 128/876

(57) ABSTRACT

A body restraint apparatus preferably includes an anchor cloth, waist straps, upper limb straps, a shoulder roll, a head support roll, lower limb straps, thigh straps, and calf straps to restrain a neonate in a frog leg position during medical procedures or transport. In some embodiments, an ankle stabilizer provides additional support for the body restraint apparatus. A CPAP restraint apparatus preferably includes an anchor cloth, forehead straps, lip straps, chin straps, and an orogastric tube holder to stabilize the CPAP tubing, the orogastric tubing, and the chin of a neonate receiving CPAP. In some embodiments, an upper limb restraint apparatus provides additional support for the CPAP restraint apparatus. The apparatus are preferably made of non-elastic baby-friendly cloth with hook and loop fasteners. The apparatus may alternatively be designed and sized for use on children or adults during medical procedures, for transport, or during administration of CPAP.

### BACKGROUND OF THE INVENTION

1. Field of the Invention

The invention pertains to the field of neonatal medicine. More particularly, the invention pertains to neonatal medical apparatus and methods of using neonatal medical apparatus.

2. Description of Related Art

Neonatal babies, or neonates, especially premature infants and sick full term infants, often require multiple medical procedures during their course of hospital stay after birth. Medical procedures, including, but not limited to, endotracheal intubation, mechanical ventilation including the administration of continuous positive airway pressure (CPAP), umbilical arterial and venous catheterization, and urinary bladder catheterization, are commonly performed on the neonates. In addition, the majority of healthy male neonates undergo circumcision. It is very important to safely restrain the neonate during a medical procedure and during neonatal transport. Improper neonatal restraint may lead to multiple medical problems, including, but not limited to, sterile surgical site contamination, medical procedure failures, a non-physiological surge in counter-regulatory hormones, accidental extubation, and predisposition to developmental dysplasia of the hip joint.

Restraints are primarily applied for several purposes: to restrain the neonate while performing one or more medical procedures, to restrain the neonate during safe transport of the sick neonate, and to secure the tubing to deliver life-saving care to the neonates.

Neonates are conventionally restrained for procedures and transport using one or more different techniques as follows: 1) applying straps to both wrists and ankles and pinning the straps to the bed using safety pins, with extended upper limbs and lower limbs; 2) placing a cloth or baby diaper across both thighs in an extended and adducted position at the hips and putting a cloth tape over the cloth or baby diaper and sticking it to either side of the bed; 3) placing the infant on a hard plastic mold in the shape of an infant and securing their extremities with hook and loop fasteners during circumcision; or 4) putting a strip of hook and loop fasteners across the forehead, chest, and thighs during transport.

The normal posture of a neonate is a result of the muscular tone of the individual. The normal posture of a full term neonate is flexion at the elbow with external rotation of the arms and abduction and external rotation of the thighs with mild flexion at the hip joint, commonly referred to as the “frog leg posture”. This frog leg posture allows the normal postnatal development of the acetabulum by deepening of the socket and growth of the labrum. Positioning of the neonate in adduction and extension at the hip joint leads to eversion of the labrum and elongation of the ligamentum teres, preventing the normal ossification of the acetabulum. Tight swaddling and conventional methods of neonatal restraint position the neonate in this abnormal position. Neonatal medical procedures may take anywhere from a few minutes to couple of hours. It is very important to restrain the neonates in their normal posture during this time. The above-mentioned conventional techniques are not baby-friendly and some may predispose a neonate to developmental dysplasia of the hip joints. The above-mentioned third technique is limited only to circumcisions and has its own multiple limitations, including, but not limited to, permanent chemical stains of the restraint device, risk of cross contamination, use of a hard surface for the fragile neonates, and most importantly difficulty to offer routine care to the neonate.

U.S. Pat. No. 4,027,869, entitled “Patient Restraint for X-ray Studies of Infants” and issued Jun. 7, 1977 to Ruiz, discloses an apparatus to immobilize an infant, particularly in connection with medical studies, such as x-ray studies, requiring a frog leg position. The apparatus is not baby-friendly in that the straps are coupled to a rigid board, which is placed under the infant and would be very uncomfortable for the infant for an extended period of time during a surgical procedure or transport of the infant. In addition, the device does not restrain the upper limbs.

U.S. Pat. No. 5,329,934, entitled “Medical Patient Restraint Device” and issued Jul. 19, 1994 to Bowman, discloses a device for restraining patients during surgical procedures such as the circumcision of infants. Individual restraining straps are provided for releasable connection to respective ones of the patient's arms and legs. Mating fasteners are fixed to the pair of support straps and the individual restraining straps such that each of the restraining straps may be releasably attached to one of the pair of support straps so as to permit the individual straps to retain the patient in a spread-eagle position on the support member without having the individual restraining straps extending across the torso of the patient.

There is a strong need for a baby-friendly, inexpensive, disposable neonatal restraint system that easily and quickly secures a neonate so that medical procedures and neonatal transport may be accomplished safely without injury to the neonate.

CPAP is given to neonates in respiratory distress, and it is very important to stabilize the CPAP tubing securely for better compliance and efficacy. CPAP tubing is conventionally secured using various methods, including, but not limited to, using four safety pins pinned to the neonatal cap just above the eyes and two rubber bands to hold the inspiratory and expiratory corrugated tubes of the CPAP device and using a strap of hook and loop fasteners to hold the inspiratory and expiratory tubing of the CPAP device to the forehead. A shoulder roll formed from folds of cloth is conventionally placed under the shoulders of the neonate receiving CPAP in order to keep the airway clear in the neonate, as the occiput is prominent in neonates, which may lead to partial airway obstruction. In addition, a roll of blanket is placed around the neonate's head during positive pressure ventilation to prevent accidental dislodging of the endotracheal tube or CPAP prongs.

In any neonate with respiratory distress needing CPAP, it is very important to administer the positive airway pressure quickly and effectively. Securing the CPAP tubing properly is very important for the following reasons: 1) to provide easy maneuverability of the neonate for the caregivers to provide routine care; 2) to prevent nasal septal erosion or perforation; and 3) to prevent CPAP failures and subjecting the neonate to invasive modes of ventilation.

The conventional method of securing CPAP tubing is not baby-friendly and may lead to complications, including, but not limited to, injury to the eye or skin with the safety pin, nasal septal erosion, nasal septal perforation, and CPAP failures due to ineffective positive pressure delivery to the neonate. In addition, a neonate on CPAP must have an orogastric tube in place to prevent gas distension of the stomach, which may lead to stomach perforation.

### SUMMARY OF THE INVENTION

In some embodiments, a body restraint apparatus includes an upper limb restraint apparatus and a lower limb restraint apparatus. The upper limb restraint apparatus includes a shoulder roll, a head support roll, and upper limb straps. The lower limb restraint apparatus includes an anchor cloth, waist straps, lower limb straps, thigh straps, and calf straps to restrain a neonate in a frog leg position during medical procedures or transport of the neonate. In other embodiments, the body restraint apparatus includes an anchor cloth, waist straps, upper limb straps, lower limb straps, thigh straps, and calf straps to restrain a neonate in a frog leg position during medical procedures or transport of the neonate. In some embodiments, an ankle stabilizer provides additional support for the body restraint apparatus. A CPAP restraint apparatus preferably includes an anchor cloth, forehead straps, lip straps, chin straps, and an orogastric tube holder to stabilize the CPAP tubing, the orogastric tubing and, the chin of a neonate receiving CPAP. In some embodiments, an upper limb restraint apparatus provides additional support for the CPAP restraint apparatus. The upper limb restraint apparatus may also be used in intubated neonates, as it prevents the neonate from dislodging the endotracheal tube. The straps are preferably made of non-elastic baby-friendly cloth. The restraints are preferably fastened using hook and loop fasteners. The apparatus may alternatively be designed and sized for use on children and adults during medical procedures, for transport, or during administration of CPAP.

### BRIEF DESCRIPTION OF THE DRAWINGS

FIG. 1a shows a waist strap of a body restraint apparatus with a first portion of a fastener on one end and a second portion of the fastener on the other end, extending from the anchor cloth under the lower back of the neonate in an embodiment of the present invention.

FIG. 1b shows the waist strap of FIG. 1a encircling the abdomen just below the umbilicus and fastened by the fastener.

FIG. 2a shows two lower limb straps of a body restraint apparatus extending from the anchor cloth at the popliteal angle on both sides, each strap ending with a thigh strap and a calf strap attached partly to each other, in an embodiment of the present invention.

FIG. 2b shows the thigh strap and calf strap of FIG. 2a encircling the lower part of the thigh and the upper part of the calf using the fasteners.

FIG. 3a shows a front view of a lower limb restraint apparatus in an embodiment of the present invention.

FIG. 3b shows a back view of the lower limb restraint apparatus of FIG. 3a.

FIG. 3c shows a front view of an ankle stabilizer apparatus in an embodiment of the present invention.

FIG. 4 shows a neonate with waist straps, thigh straps, and calf straps of the lower limb restraint apparatus of FIG. 3a in place in an embodiment of the present invention.

FIG. 5 shows a neonate with the lower limb restraint apparatus of FIG. 3a and the ankle stabilizer apparatus of FIG. 3c in place in an embodiment of the present invention.

FIG. 6a shows a front view of a limb restraint apparatus in an embodiment of the present invention.

FIG. 6b shows a back view of the limb restraint apparatus of FIG. 6a.

FIG. 7 shows a neonate with upper limb straps, waist straps, thigh straps, and calf straps of the limb restraint apparatus of FIG. 6a in place in an embodiment of the present invention.

FIG. 8a shows a front view of an upper limb restraint apparatus with shoulder roll and head support roll in an embodiment of the present invention.

FIG. 8b shows a side view of the upper limb restraint apparatus of FIG. 8a.

FIG. 9a shows the upper limb restraint apparatus of FIG. 8a under a neonate with the two upper limb straps extending from the anchor cloth under the upper back of the neonate.

FIG. 9b shows the upper limb straps of FIG. 9a encircling the wrists and fastened by the fasteners.

FIG. 10a shows a forehead strap of a CPAP restraint apparatus extending from an anchor cloth positioned behind the head of a neonate in an embodiment of the present invention.

FIG. 10b shows the CPAP restraint apparatus of FIG. 10a with the forehead strap fastened around the forehead of the neonate.

FIG. 11 shows the CPAP restraint apparatus of FIG. 10b with the two lip straps fastened around the CPAP tubing to hold the tubing firmly above the upper lip.

FIG. 12 shows the CPAP restraint apparatus of FIG. 11 with the chin strap fastened under the chin to hold the chin when the CPAP is in place and the orogastric tube holder securing the orogastric tube.

FIG. 13a shows a front view of a CPAP restraint apparatus in an embodiment of the present invention.

FIG. 13b shows a back view of the CPAP restraint apparatus of FIG. 13a.

FIG. 14 shows a neonate fully restrained by the lower limb restraint apparatus of FIG. 3a, the upper limb restraint apparatus of FIG. 8a, and the CPAP restraint apparatus of FIG. 13a.

### DETAILED DESCRIPTION OF THE INVENTION

In a preferred embodiment, a neonatal body restraint apparatus keeps the neonate in its usual baby-friendly position during medical procedures without hindering the medical procedures and allows safe neonatal transport. The neonatal body restraint apparatus preferably includes multiple bands of soft, firm cloth and baby-friendly fasteners. The cloth is baby-friendly and non-elastic. Baby-friendly cloth may be any material that minimally irritates neonates' skin. In some embodiments, the baby-friendly, non-elastic cloth material is cotton. In some embodiments, the fasteners are hook and loop fasteners. In these embodiments, the hook and loop fasteners are preferably hospital-grade. In some embodiments, a rectangular strip of cloth (“anchor cloth”) is used as an anchor from which a waist strap and two lower limb straps extend out to keep the neonate in its normal posture. The waist strap preferably encircles the abdomen just below the umbilicus and is attached in the front using fasteners. The lower limb straps extend from the base of the anchor cloth and extend to the popliteal fossa on either side of the neonate. Two cloth straps encircle the lower part of the thigh and the upper part of the calf These lower limb straps prevent flexion at the hip joint, leaving the neonate in the normal frog leg position.

For large, vigorous neonates, an ankle stabilizer apparatus is used in addition to the neonatal body restraint apparatus. Fasteners around the ankles of the neonate provide additional baby-friendly restraint. The ankle stabilizer apparatus is preferably used when the above-described neonatal body restraint apparatus is not strong enough to constrain the neonate to the normal frog leg position.

In some embodiments, the upper limb straps extend from the mid-lower part of the shoulder roll of an upper limb restraint apparatus. In other embodiments, the upper limb straps extend from either side of the anchor cloth, just above the waist strap. The upper limb straps fasten both wrists.

A neonate's weight is used to control the flexion at the hip joint. The length between the mid sacral and the popliteal fossa increases as the hip joint flexion increases, due to the natural contours of the body. The anchor cloth is placed under the sacrum, and a fixed, non-elastic strap extends from the sacral region to the popliteal region and is securely held by the lower limb straps to prevent the flexion at the hip joint. In addition, the neonate's weight prevents the flexion of the upper limb when the wrist is held with the upper limb strap extending from the sacral region in some embodiments and from the shoulder blade region in other embodiments.

The ¾″-thick shoulder roll is made of folds of baby-friendly non-elastic cloth material or baby-friendly firm foam material. The head support roll is also made of baby-friendly cloth material or baby-friendly firm foam material and its height gradually increases from below the ear level, along the sides of the head to the top of the head. The anchor cloth and the straps of the body restraint apparatus are preferably made of a baby-friendly non-elastic cloth material. The fasteners are also preferably baby-friendly. In some embodiments, the fasteners are hook and loop fasteners.

Neonatal size varies significantly based on their gestational age and weight, and the body restraint apparatus is preferably sized to fit the neonate. A body restraint apparatus is preferably used on a baby weighing in the range of 500 grams to 4500 grams. The body restraint apparatus is preferably provided in five sizes based primarily on the weight of the neonate or infant: extremely small (birth weight less than one kg), small (birth weight between one and two kg), medium (birth weight between two and three kg), large (birth weight three to four kg), and extra large (greater than four kg).

The following measurements are preferably collected on the neonate: 1) the abdominal circumference one inch below the umbilicus; 2) the distance from the mid-scapular region to the wrist when the upper limb is flexed and externally rotated; 3) the wrist circumference; 4) the distance from the mid sacrum to the popliteal fossa, when the thigh is slightly flexed, abducted, and externally rotated, with a hip extension angle (the angle between the bed surface and neonate's thigh) of 20 degrees and a hip abduction angle (the angle between the inner thigh and midline trunk axis) of 40 degrees, the angles preferably being measured using a goniometer; 5) the lower thigh circumference; 6) the upper leg circumference; 7) the ankle circumference; and 8) the distance from one shoulder to the other shoulder along the posterior side of the ears and the top of the head.

In a preferred method, the following steps are involved in securing the body restraint apparatus:

1. Determine or approximate the weight of the baby.

2. Select a restraint apparatus of the appropriate size based on the weight of the baby.

3. Place the restraint apparatus on the neonatal bed or other baby-friendly surface.

4. Place the baby on the shoulder roll with the wrist straps extending on either side and the head support roll around the posterior aspect of the baby's head and the lower back on the anchor cloth of the lower limb restraint apparatus in the frog leg position.

5. Encircle and fasten the waist strap just below the umbilicus of the baby.

6. Encircle and fasten the upper limb straps around the wrists of the baby.

7. Encircle and fasten each thigh strap around the lower part of the thigh.

8. Encircle and fasten each calf strap around the upper part of the calf

9. If the baby is very vigorous or large, fasten the ankles to the straps of the ankle stabilizer and tuck the ankle stabilizer in on either side of the bed.

In embodiments where hook and loop fasteners are used, to prevent the chafing of the neonate's skin, the hook portion is preferably narrower than the loop portion of each fastener. In this way, when fastened, the full width of the hook portion is fully covered by the loop portion. In some embodiments, the hook portion is about half the width of the loop portion. In addition, the hook and loop fastener is preferably securely stitched to a firm baby-friendly cloth. In some embodiments, the fasteners are stitched to a cloth strap backing. In other embodiments, the fasteners are only stitched to the end of the cloth strap and extend from the strap without a cloth strap backing. The body restraint apparatus is preferably low-cost and disposable.

The body restraint apparatus may alternatively be tailored for use either in pediatric or adult humans or small to medium sized pets, while performing any medical or surgical procedures. The body restraint apparatus may alternatively be meshed into infant clothing, especially three- to four-month old babies, and used to reduce the risk of sudden infant death syndrome (SIDS). The principle behind SIDS reduction is to prevent the baby from rotating to a prone position from a supine position.

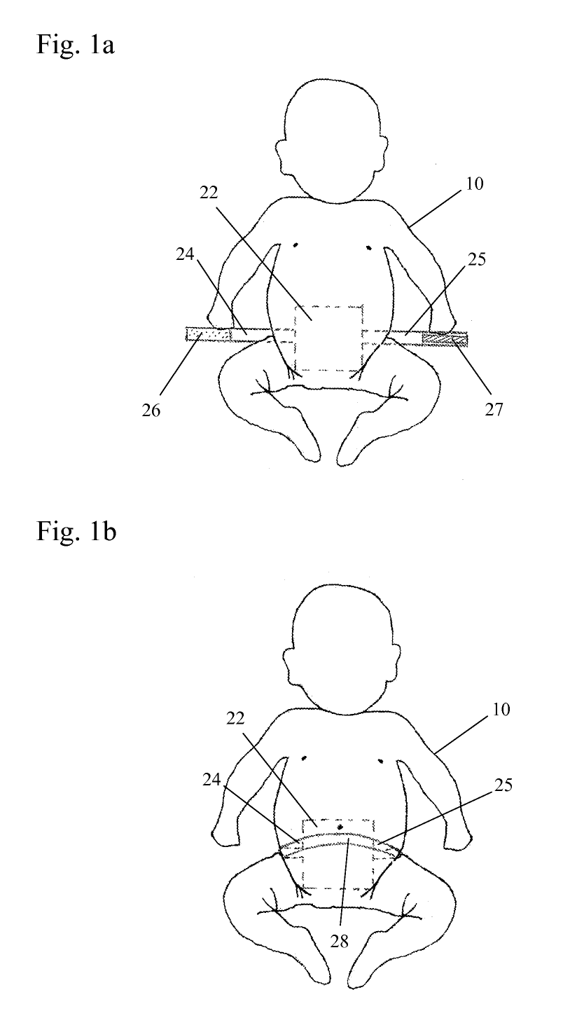

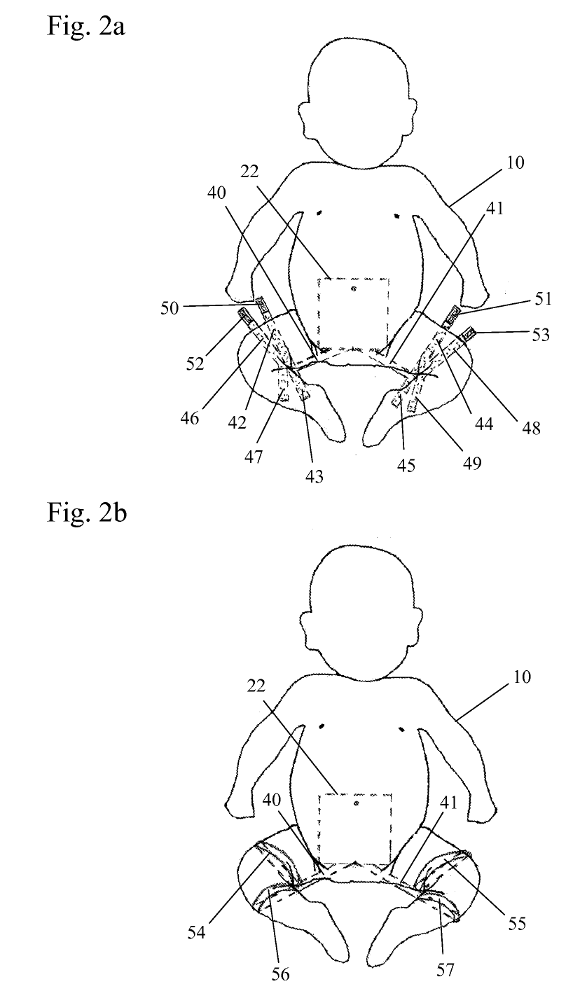

FIGS. 1a through 2b show stages of applying a body restraint apparatus to a neonate. The neonate 10 is placed on the body restraint apparatus with the anchor cloth 22 centered in the lower back/upper buttocks region with the waist straps 24, 25 extending from the sides of the anchor cloth 22 at the neonate's waist, as shown in FIG. 1a. One waist strap 24 includes loops 26 of a hook and loop fastener on the end of the waist strap 24. The other waist strap 25 includes a hook area 27 of the hook and loop fastener on the end of the waist strap 25. The hook and loop fastener is then used to fasten the waist straps 24, 25 around the waist of the neonate 10, as shown in FIG. 1b. The waist straps 24, 25 form a waist restraint 28 to hold the anchor cloth 22 in position behind the neonate 10. The weight of the neonate 10 on the anchor cloth 22 also works to prevent the anchor cloth 22 from shifting with respect to the neonate 10.

With the waist straps 24, 25 fastened around the waist of the neonate 10, the lower limb straps 40, 41 extend at an angle from the bottom of the anchor cloth 22 toward the neonate's knees, as shown in FIG. 2a. In some embodiments, the angle is about 40° such that the angle formed between the two lower limb straps is about 80°. Thigh straps 42, 43, 44, 45 and calf straps 46, 47, 48, 49 extend from each of the lower limb straps 40, 41. For each pair of thigh straps, one thigh strap 42, 44 has a hook area 50, 51, respectively, to fasten to a loop area on the corresponding thigh strap 43, 45. For each pair of calf straps, one calf strap 46, 48 has a hook area 52, 53, respectively, to fasten to a loop area on the corresponding calf strap 47, 49. The thigh straps 42, 43, 44, 45 and calf straps 46, 47, 48, 49 fasten around the lower thigh and upper calf, respectively, of the neonate, as shown in FIG. 2b. The thigh straps 42, 43, 44, 45 and calf straps 46, 47, 48, 49, when fastened, form thigh restraints 54, 55 and calf restraints 56, 57, respectively, that provide little or no resistance with the legs in the frog leg position but limit movement of the legs away from the frog leg position.

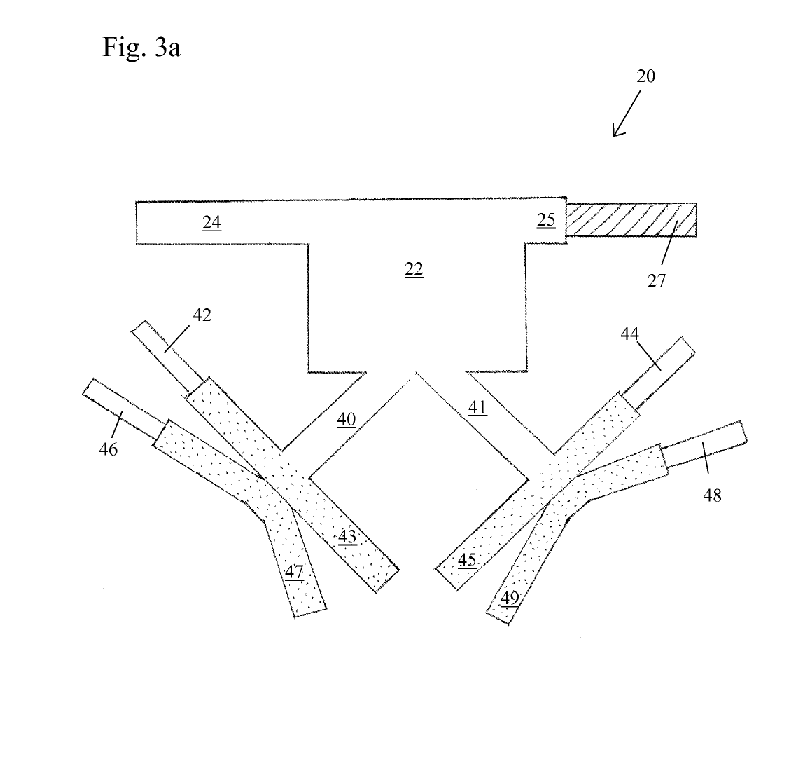

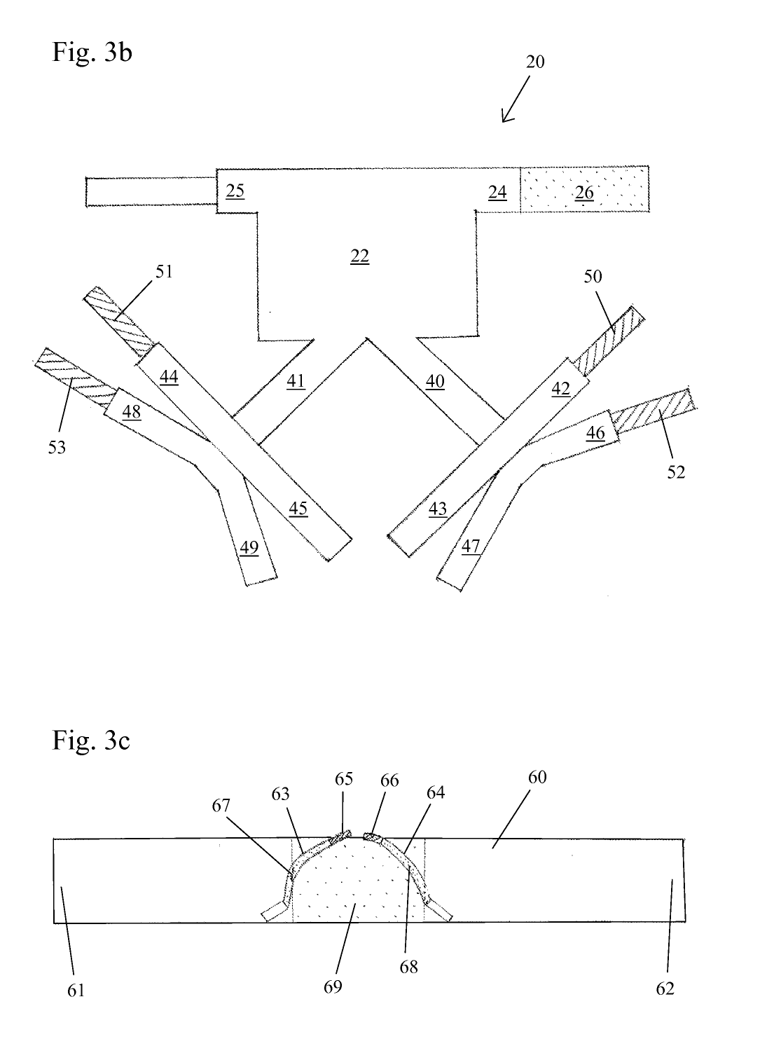

FIG. 3a and FIG. 3b show a front view and a back view, respectively, of a body restraint apparatus 20 in an embodiment of the present invention. Striped areas represent areas covered with hook fasteners and dotted areas represent areas covered with loop fasteners. For the waist straps 24, 25, the thigh straps 42, 43, 44, 45, and the calf straps 46, 47, 48, 49, the hook areas and loop areas are on opposite straps, with one being on the front of the strap and the other being on the back of the strap. Alternatively, either area may be located on the front of the strap, and either area may be located on the left strap within the spirit of the present invention. FIG. 4 shows the neonate 10 fully constrained in the frog leg position by the body restraint apparatus 21.

FIG. 3c shows a front view of an ankle stabilizer apparatus in an embodiment of the present invention. Striped areas represent areas covered with hook fasteners and dotted areas represent areas covered with loop fasteners. For the ankle straps 63, 64, the hook areas 65, 66 are on the ends of the straps 63, 64 and loop areas 67, 68 are on lower parts of the straps 63, 64. A loop area 69 is also located in a central portion of the ankle anchor 60 of the ankle stabilizer apparatus. The hook areas 65, 66 and loop areas 67, 68 are preferably both located on the front side of the ankle straps 63, 64 but may be located on opposite sides or on the back side within the spirit of the present invention. The ends 61, 62 of the ankle anchor 60 may also have a hook area or a loop area to fasten to the support structure on which the neonate is lying.

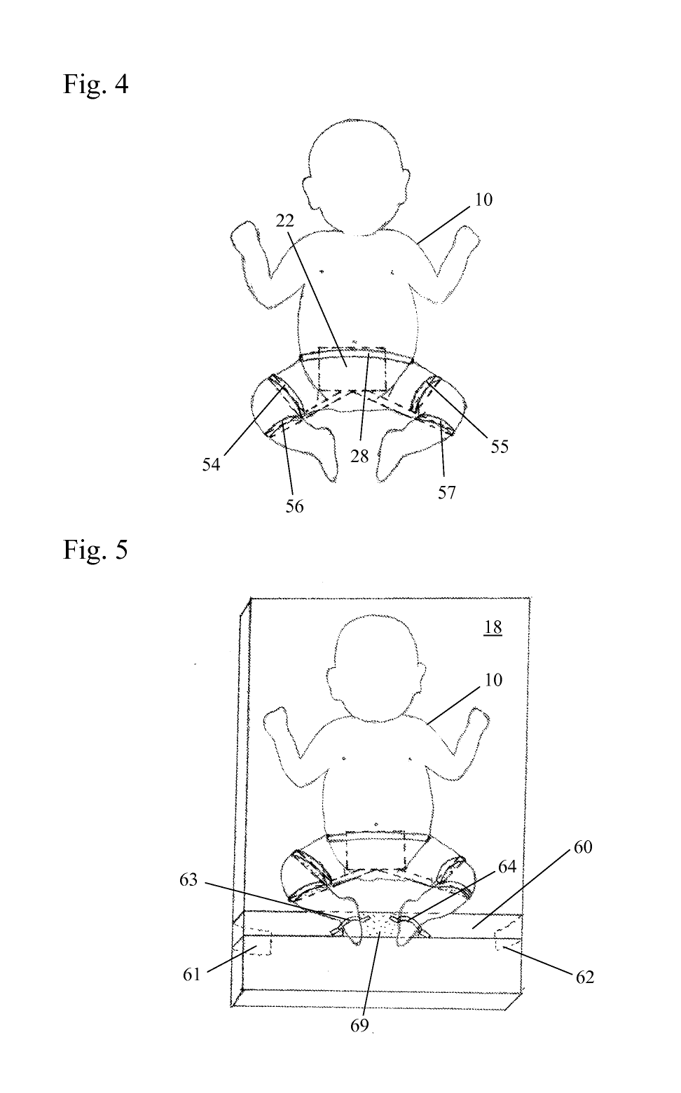

FIG. 5 shows the neonate 10 being further constrained by the ankle stabilizer apparatus. The ends 61, 62 of the ankle anchor 60 extend over the sides of the support structure 18 and tuck under the support structure 18. The ankle straps 63, 64 have been placed over the ankles of the neonate 10 in the frog leg position with the hook areas of the straps 63, 64 being fastened to the loop area 69 on the ankle anchor 60 to restrain the feet of the neonate 10. The hook areas 65, 66 may alternatively be fastened to the loop areas 67, 68 of the ankle straps 63, 64 to stabilize the ankles of the neonate 10 within the spirit of the present invention. The ankle stabilizer apparatus provides additional support to the body restraint apparatus, which may be needed in the case of a large or highly-active neonate 10.

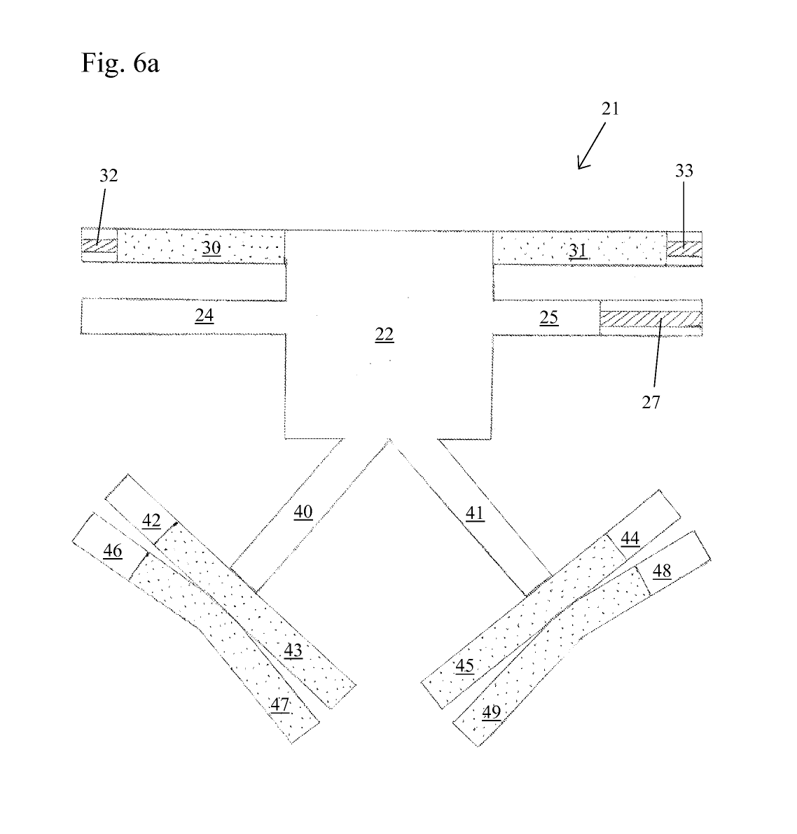

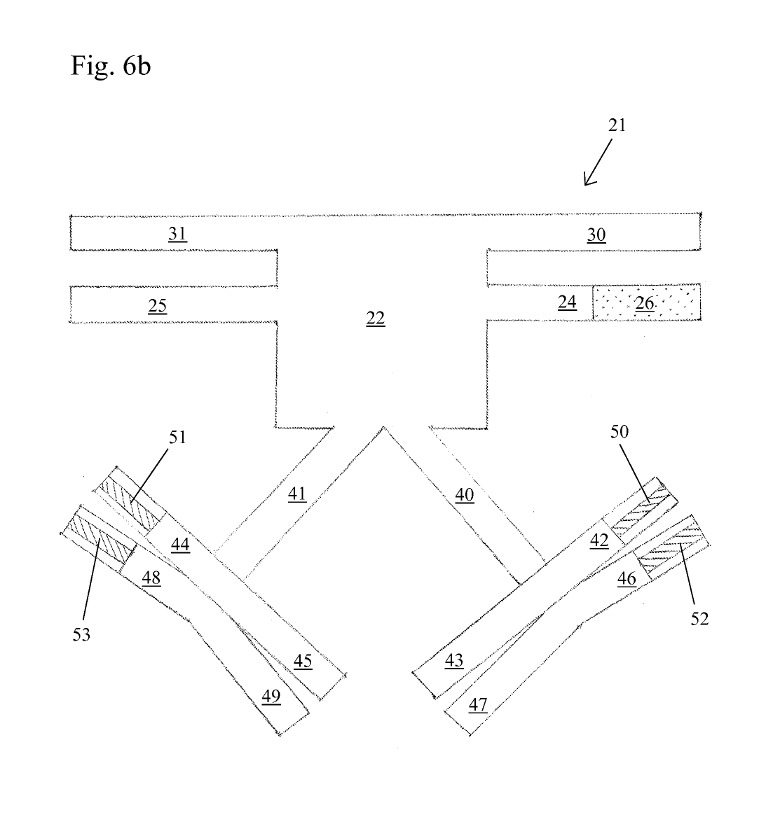

FIG. 6a and FIG. 6b show a front view and a back view, respectively, of a limb restraint apparatus 21 in an embodiment of the present invention. Striped areas represent areas covered with hook fasteners and dotted areas represent areas covered with loop fasteners. For the waist straps 24, 25, the thigh straps 42, 43, 44, 45, and the calf straps 46, 47, 48, 49, the hook areas and loop areas are on opposite straps, with one being on the front of the strap and the other being on the back of the strap. Alternatively, either area may be located on the front of the strap, and either area may be located on the left strap within the spirit of the present invention. For the upper limb straps 30, 31, the hook areas 32, 33 are on the ends of the straps 30, 31 and the loop areas are farther down on the strap on the opposite side of the strap, but either area may be on the front of the strap within the spirit of the present invention. FIG. 7 shows the neonate 10 fully constrained in the frog leg position by the limb restraint apparatus 21.

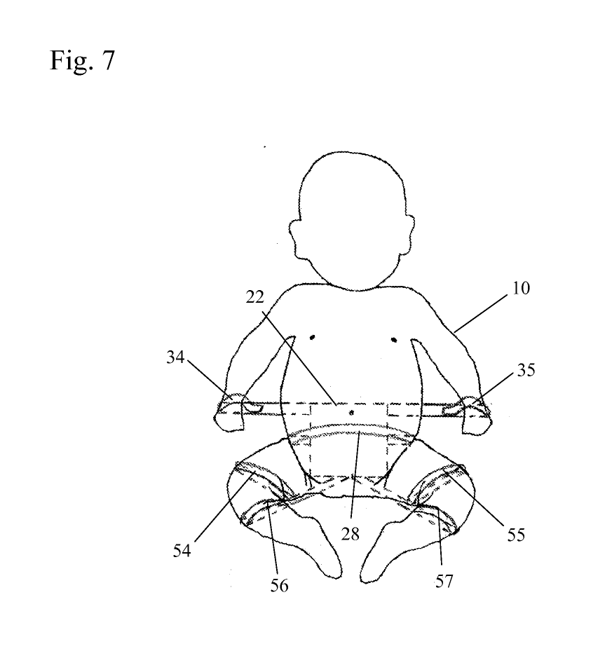

FIG. 8a and FIG. 8b show a top view and a side view, respectively, of an upper limb restraint apparatus 110. A shoulder roll 114 supports the shoulders of the neonate. A head support roll 112 extends from the top of the shoulder roll 114 to support the head of the neonate. The head support roll 112 is thicker toward the top, as seen in FIG. 8b. The two head support roll 112 attachment point straps to the shoulder roll 114 are preferably adjustable so that the head support roll 112 conforms to the head size of the neonate. In some embodiments, each attachment point includes a ring to adjust the strap lengths. Two upper limb straps 115, 116 extend outward in opposite directions from a lower portion of the shoulder roll 114 at a slightly upward angle toward the head support roll 112. Striped areas represent areas covered with hook fasteners and dotted areas represent areas covered with loop fasteners. For the upper limb straps 115, 116, the hook areas 117, 118 are on the ends of the straps 115, 116 and the loop areas are farther down on the strap on the opposite side of the strap, but either area may be on the front of the strap within the spirit of the present invention.

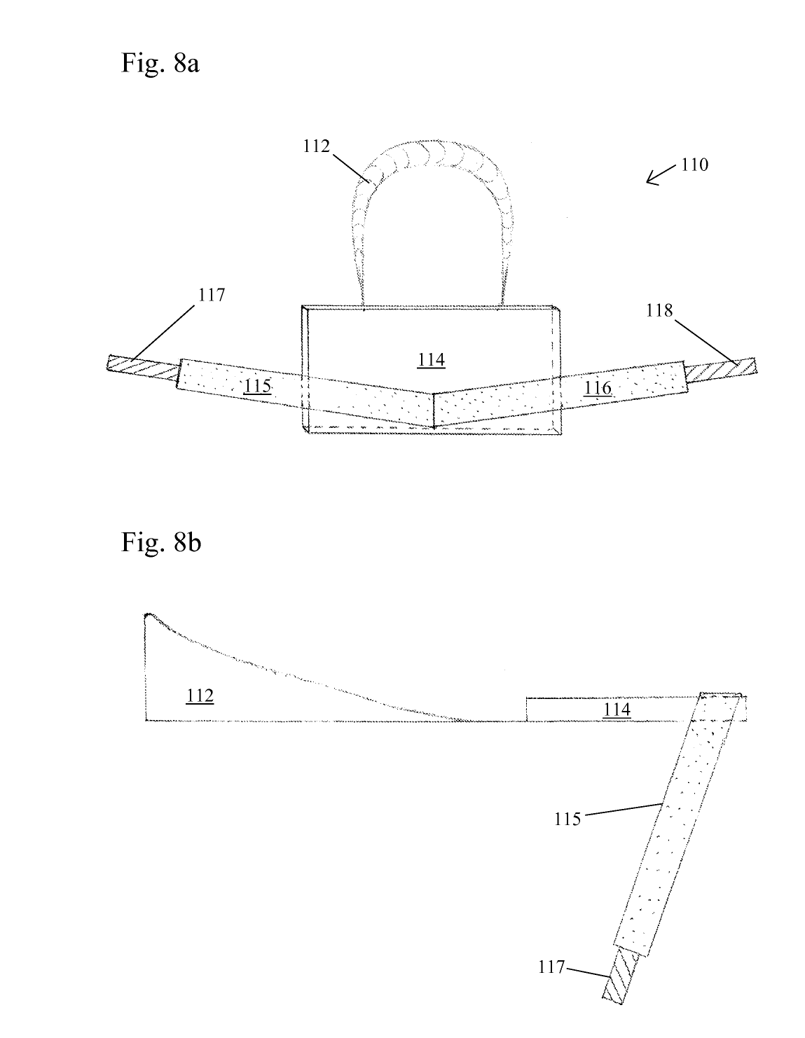

FIGS. 9a and 9b show stages of applying an upper limb restraint apparatus. The neonate 10 is placed on the upper limb restraint apparatus with the shoulder roll 114 under both the shoulder blades and the head support roll 112 following the sides of the baby's head behind the ears and the top of the head. The upper limb straps 115, 116 extend from the mid-scapular region of the shoulder roll 114 in line with the wrists of the neonate, as shown in FIG. 9a. The upper limb straps 115, 116 are fastened around the wrists of the neonate 10 to form wrist restraints 119, 120, as shown in FIG. 9b. The upper limb straps 115, 116 are preferably fastened tightly enough to prevent the hands from slipping out of the fastened straps but loosely enough to allow full circulation to the hands. The upper limb straps 115, 116, when fastened, limit movement of the arms without constraining the arms to an unnatural position.

In some embodiments of the present invention a CPAP restraint apparatus secures the CPAP tubing being used on a neonate in a quick and baby-safe manner. The CPAP restraint apparatus allows easy maneuverability for routine care, reminds the caregiver to place the orogastric tube construct, and prevents pressure on the nasal septum from the tubing. The neonatal CPAP tubing restraint apparatus preferably includes multiple bands of soft, firm cloth, baby-friendly fasteners, a soft plastic rectangle, and sticky tape with a red tab. In some embodiments, the baby-friendly fasteners are hook and loop fasteners. A rectangular strip of cloth (anchor cloth) is preferably used as an anchor, from which a forehead strap, two lip straps, and a chin strap with an orogastric tube holder extend to secure the CPAP tubing and an orogastric tube.

The forehead strap encircles the forehead just above the eyebrows and is attached in the front using the fastener, which also holds the CPAP inspiratory and expiratory tubing. Lip straps extend from either side of the anchor cloth and lie just below the ear lobes when fastened to the CPAP tubing. The lip straps fasten the CPAP tubing and hold the tubing firmly above the upper lip. The chin strap extends perpendicularly to the lower side of the lip straps. The chin strap holds the chin in place when the CPAP is initiated. The orogastric tube holder with a red tab extends perpendicularly to the lower side of the left lip strap. The orogastric tube holder holds the orogastric tube in place and the red tab serves as a visual reminder to insert the orogastric tube. This CPAP restraint apparatus helps avoid the use of any sticky tape on the neonate's delicate skin, thereby preventing abrasion or tape reaction.

In some embodiments, a shoulder roll has wrist straps extending to and wrapping either wrist from the mid-scapular region. The shoulder roll prevents partial or complete obstruction of the neonate's airway. In other embodiments, a limb restraint apparatus is used to restrain the wrists of the neonate in combination with the use of a CPAP restraint apparatus to prevent the neonate from accidentally dislodging the CPAP airway tubing. In other embodiments, wrist straps extending from the anchor cloth of the body restraint apparatus are used to restrain the wrists of the neonate in combination with the use of a CPAP restraint apparatus to prevent the neonate from accidentally dislodging the CPAP airway tubing.

The anchor cloth and the straps of the CPAP restraint apparatus and the shoulder roll and head support roll of the upper limb restraint apparatus are preferably made of a baby-friendly non-elastic cloth material. The fasteners are also preferably baby-friendly. In some embodiments, the fasteners are hook and loop fasteners. The orogastric tube holder is preferably made of a soft plastic rectangular strip and sticky tape with a red paper tab.

Neonatal size varies significantly based on their gestational age and weight, and the CPAP restraint apparatus is preferably sized to fit the neonate. The CPAP restraint apparatus is preferably used on babies weighing in the range of 500 grams to 4500 grams. The CPAP restraint apparatus is preferably provided in five sizes based primarily on the weight of the neonate: extremely small (birth weight less than one kg), small (birth weight between one and two kg), medium (birth weight between two and three kg), large (birth weight three to four kg), and extra large (greater than four kg).

The following measurements are preferably collected on the neonate: 1) the head circumference; 2) the ear-to-ear distance in the front; 3) the eye brow to upper lip distance; 4) the distance from the ear lobe to the angle of the mouth on either side; and 5) the distance from the left angle of the mouth along the chin to the right angle of the mouth.

In a preferred method, the following steps are involved in securing the CPAP restraint apparatus:

1. Determine or approximate the weight of the baby.

2. Select a CPAP restraint apparatus of the appropriate size based on the weight of the baby.

3. Place the CPAP restraint apparatus on the neonatal bed or other baby-friendly surface, and place the shoulder roll with wrist straps on the neonatal bed or other baby-friendly surface.

4. Place the baby's head on the CPAP restraint apparatus.

5. Encircle and fasten the forehead strap just above the eyebrow with the CPAP tubing in between the forehead strap fastener.

6. Encircle and fasten the lip straps on either side around the CPAP tubing above the upper lip, holding the CPAP tubing firmly.

7. Encircle and fasten the chin straps under the chin to keep the mouth closed, thereby facilitating effective delivery of positive pressure into the lungs of the baby.

8. Secure the orogastric tube using the sticky tape with the tab against the soft plastic strip.

9. Encircle and fasten the wrist straps if the neonate is highly-active to prevent the neonate from dislodging the CPAP airway tubing.

In embodiments where hook and loop fasteners are used, to prevent the chafing of the neonate's skin, the hook portion is preferably thinner than the loop portion of each fastener. In this way, when fastened, the full width of the hook portion is fully covered by the loop portion. In some embodiments, the hook portion is about half the width of the loop portion. In addition, the hook and loop fastener is preferably securely stitched to a firm baby-friendly cloth. The CPAP restraint apparatus is preferably low-cost and disposable.

This CPAP restraint apparatus may alternatively be tailored for use either in pediatric or on adult humans while using CPAP.

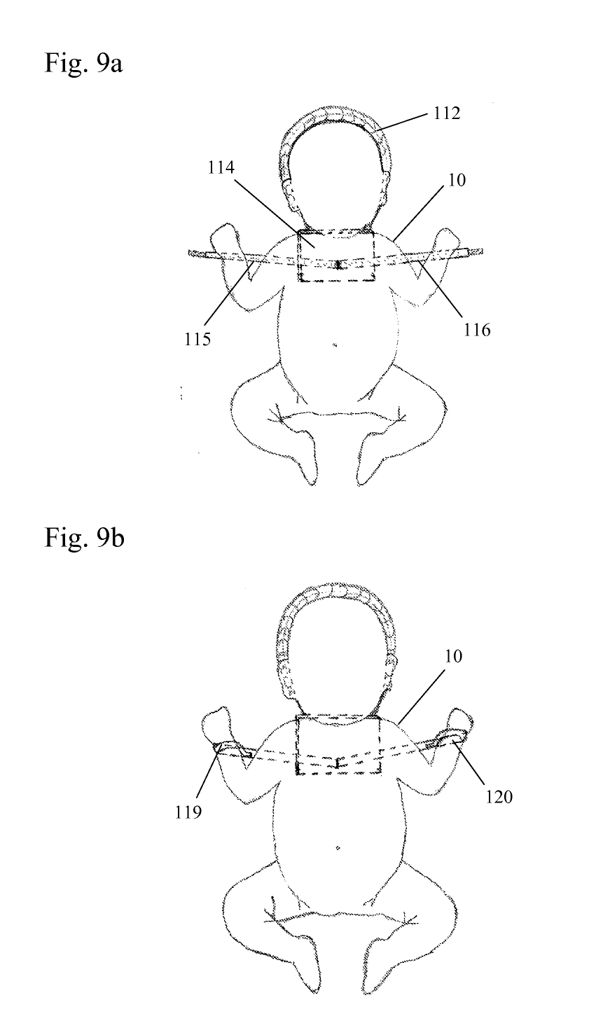

FIG. 10a through FIG. 12 show stages of applying a CPAP restraint apparatus to a neonate. The neonate 10 is placed on the CPAP restraint apparatus with the anchor cloth 82 centered behind the head 14 of the neonate and the forehead straps 84, 85 extending from the top of the sides of the anchor cloth 82 at the neonate's forehead, as shown in FIG. 10a. One forehead strap 85 includes a loop area 87 of a hook and loop fastener on the end of the forehead strap 85. The other forehead strap 84 includes a hook area 86 of the hook and loop fastener on the end of the forehead strap 84. The hook and loop fastener is then used to fasten the forehead straps 84, 85 around the forehead of the neonate 10, as shown in FIG. 10b. The forehead straps 84, 85 form a forehead restraint 88 to hold the anchor cloth 82 in position behind the neonate 10. The forehead restraint 88 also holds the CPAP tubings 71, 72 in place. The weight of the neonate's head 14 on the anchor cloth 82 also works to prevent the anchor cloth 82 from shifting with respect to the neonate 10.

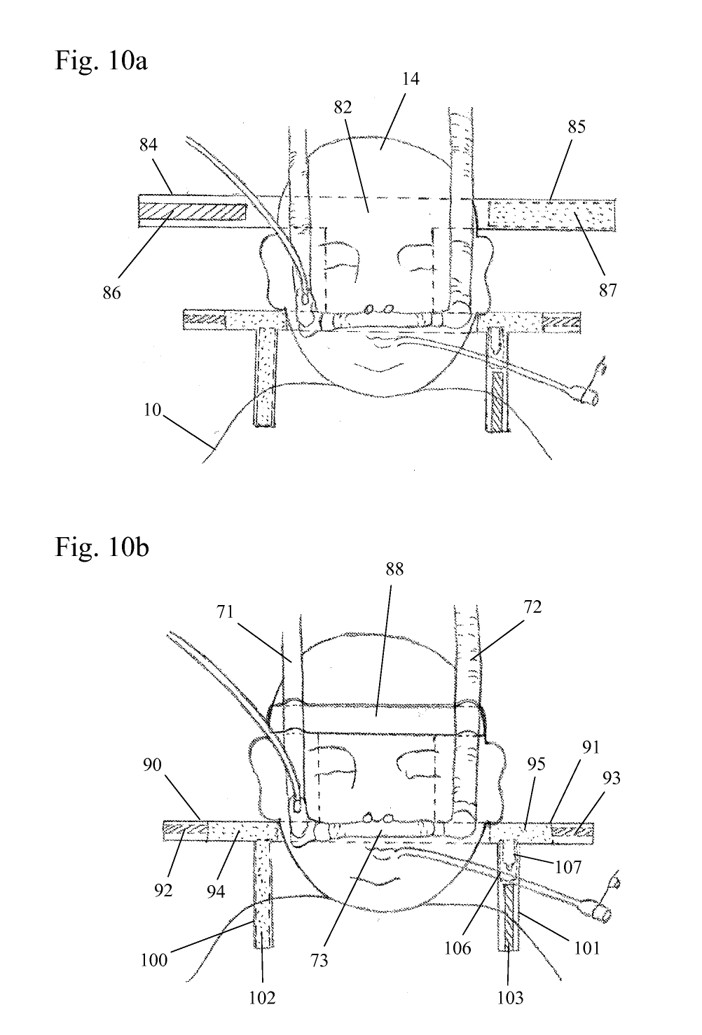

The upper lip straps 90, 91 extend from the sides of the anchor cloth 82 to align with the CPAP upper lip tubing 73, as shown in FIG. 10b. Each upper lip strap 90, 91 includes a hook area 92, 93 and a loop area 94, 95 of a hook and loop fastener. The hook and loop fasteners are then used to fasten the upper lip straps 90, 91 around the CPAP tubing at the tubing elbows between the upper CPAP tubing 71, 72 and the CPAP upper lip tubing 73, as shown in FIG. 11. The upper lip straps 90, 91 form upper lip restraints 96, 97 to hold the CPAP tubing 73 in place.

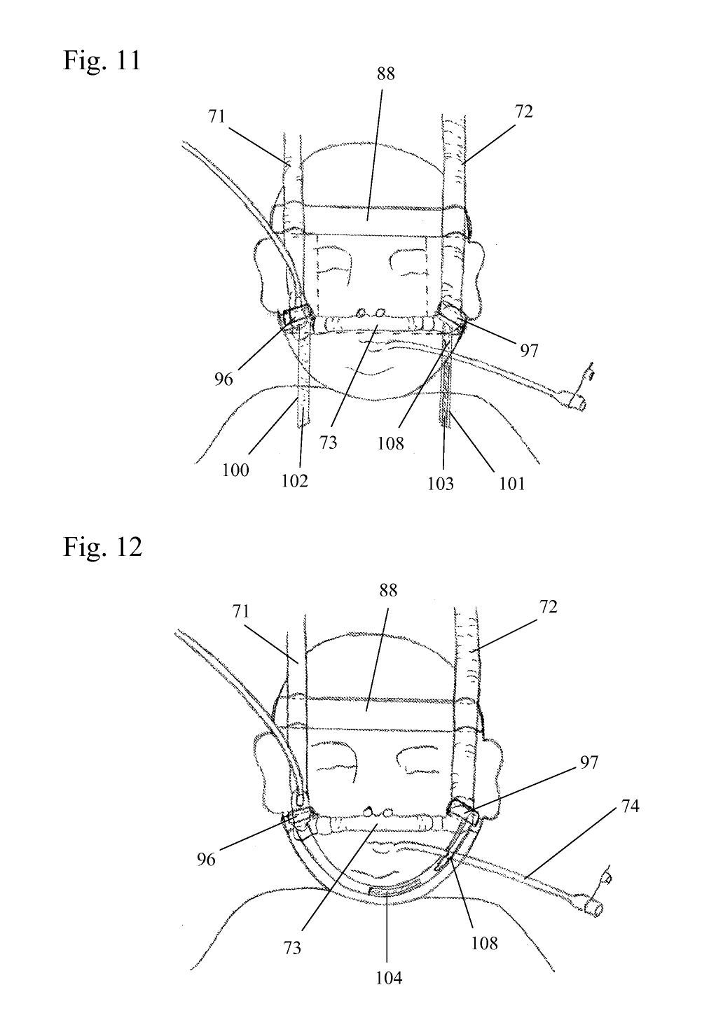

The chin straps 100, 101 extend downward from the upper lip straps 90, 91, as shown in FIG. 10b and FIG. 11. With the upper lip straps 90, 91 fastened, as shown in FIG. 11, the chin straps 100, 101 extend downward from the cheeks of the neonate 10. The loop area 102 on the first chin strap 100 and the hook area 103 on the second chin strap 101 are fastened together under the chin of the neonate to form the chin restraint 104, as shown in FIG. 12. An adhesive tab 106 and a soft plastic strip 107 form an orogastric tube holder 108 holding the orogastric tubing 74 of the CPAP apparatus in position. The adhesive tab 106 is preferably a bright color, such as bright red, to remind the attendant to insert and attach the orogastric tubing 74.

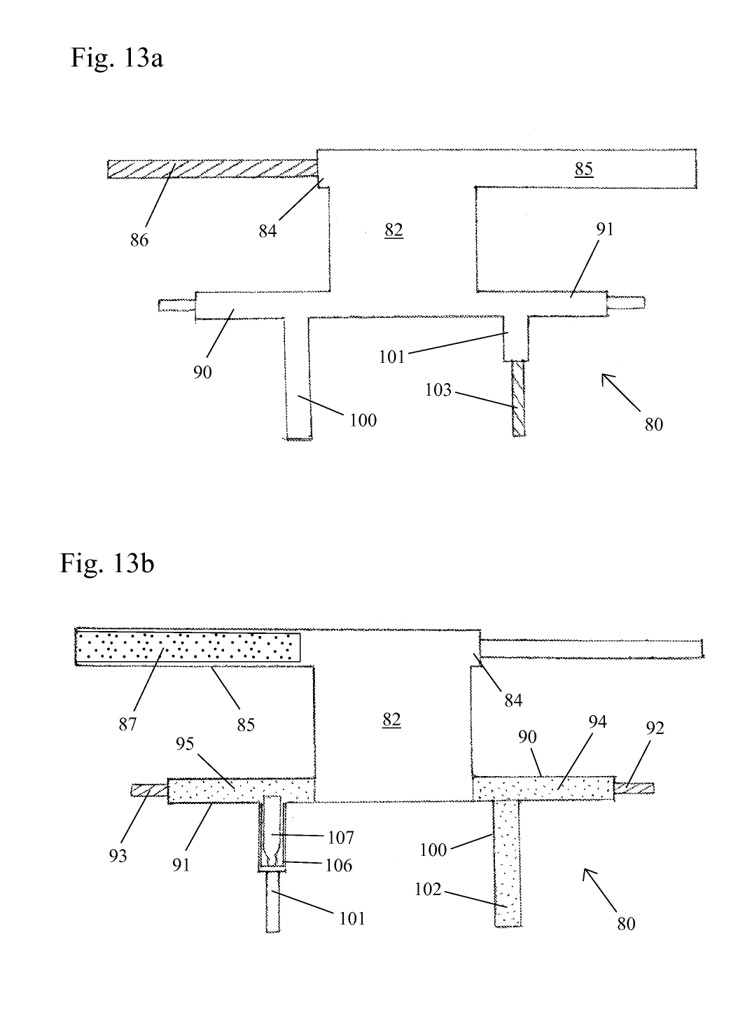

FIG. 13a and FIG. 13b show a front view and a back view, respectively, of a CPAP restraint apparatus 80 in an embodiment of the present invention. Striped areas represent areas covered with hook fasteners and dotted areas represent areas covered with loop fasteners. For the forehead straps 84, 85 and the chin straps 100, 101, the hook areas and loop areas are on opposite straps, with one being on the front of the strap and the other being on the back of the strap, but either may be on the front and either may be on the left strap within the spirit of the present invention. For the upper lip straps 90, 91, the hook areas 92, 93 are toward the ends of the straps 90, 91 and the loop areas 94, 95 are farther down on the strap on the back side of the strap, but either or both areas may be on the front of the strap within the spirit of the present invention. FIG. 12 shows the CPAP restraint apparatus fully applied to the neonate 10 and the CPAP.

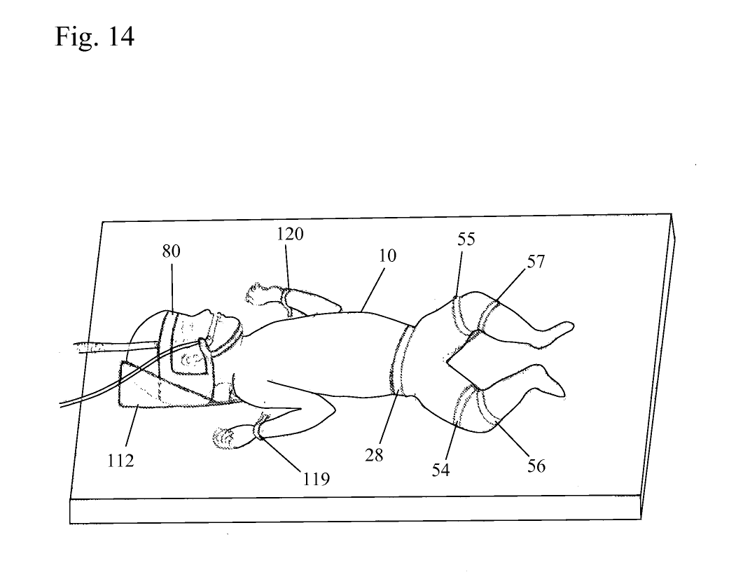

FIG. 14 shows a neonate 10 being administered CPAP with the waist restraint 28, thigh restraints 54, 55, calf restraints 56, 57, wrist restraints 119, 120, and the CPAP restraint apparatus 80 in place.

Although hook and loop fasteners are shown as the fasteners in the illustrated embodiments, other fasteners may alternatively be used within the spirit of the present invention. Although specific locations on the straps are shown for hook areas and loop areas in the illustrated embodiments, other locations and other combinations of locations for the hook areas and the loop areas may be used within the spirit of the present invention, including, but not limited to, switching the hook area locations with the loop area locations.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A body restraint apparatus comprising:
an anchor cloth having a first side edge, a second side edge opposite the first side edge, and a bottom edge;
a first waist strap extending from the first side edge of the anchor cloth;
a first waist strap fastener portion located on a distal portion of the first waist strap;
a second waist strap extending from the second side edge of the anchor cloth;
a second waist strap fastener portion, complementary to the first waist strap fastener portion, located on a distal portion of the second waist strap;
a first lower limb strap extending at a first angle from the bottom edge of the anchor cloth;
a second lower limb strap extending at a second angle from the bottom edge of the anchor cloth;
a first thigh strap and a second thigh strap extending sideways in opposite directions from a distal end of the first lower limb strap;
a first thigh strap fastener portion located on a distal portion of the first thigh strap;
a second thigh strap fastener portion, complementary to the first thigh strap fastener portion, located on a distal portion of the second thigh strap;
a third thigh strap and a fourth thigh strap extending sideways in opposite directions from a distal end of the second lower limb strap;
a third thigh strap fastener portion located on a distal portion of the third thigh strap; and
a fourth thigh strap fastener portion, complementary to the third thigh strap fastener portion, located on a distal portion of the fourth thigh strap;
wherein the anchor cloth, the waist straps, the lower limb straps, and the thigh straps are sized such that, with the anchor cloth located on a lower back of a person and the person in a frog leg position, the first waist strap fastener portion and the second waist strap fastener portion fasten together to form a waist restraint around a waist of the person, the first thigh strap fastener portion and the second thigh strap fastener portion fasten together to form a first thigh restraint around a first lower thigh of the person, and the third thigh strap fastener portion and the fourth thigh strap fastener portion fasten together to form a second thigh restraint around a second lower thigh of the person.

2. The body restraint apparatus of claim 1 further comprising:
a first calf strap and a second calf strap extending in opposite directions and coupled to a distal side of the first thigh strap;
a first calf strap fastener portion located on a distal portion of the first calf strap;
a second calf strap fastener portion, complementary to the first calf strap fastener portion, located on a distal portion of the second calf strap;
a third calf strap and a fourth calf strap extending in opposite directions and coupled to a distal side of the second thigh strap;
a third calf strap fastener portion located on a distal portion of the third calf strap; and
a fourth calf strap fastener portion, complementary to the third calf strap fastener portion, located on a distal portion of the fourth calf strap;
wherein the calf straps are sized such that, with the anchor cloth located on the lower back of the person and the person in a frog leg position, the first calf strap fastener portion and the second calf strap fastener portion fasten together to form a first calf restraint around a first upper calf of the person, and the third calf strap fastener portion and the fourth calf strap fastener portion fasten together to form a second calf restraint around a second upper calf of the person.

3. The body restraint apparatus of claim 1 further comprising an upper limb restraint apparatus comprising:
a shoulder roll;
a head support roll extending from a top portion of the shoulder roll;
a first wrist strap extending from a bottom portion of the shoulder roll;
a first wrist strap fastener portion located on a first portion of the first wrist strap;
a second wrist strap fastener portion, complementary to the first wrist strap fastener portion, located on a second portion of the first wrist strap;
a second wrist strap extending from the bottom portion of the shoulder roll;
a third wrist strap fastener portion located on a first portion of the second wrist strap;
a fourth wrist strap fastener portion, complementary to the third wrist strap fastener portion, located on a second portion of the second wrist strap;
wherein the wrist straps are sized such that, with the shoulder roll located under the upper back and shoulders of the person, the first wrist strap fastener portion and the wrist thigh strap fastener portion fasten together to form a first wrist restraint around a first wrist of the person and the third wrist strap fastener portion and the fourth wrist strap fastener portion fasten together to form a second wrist restraint around a second wrist of the person.

4. The body restraint apparatus of claim 1, wherein the person is a neonate.

5. The body restraint apparatus of claim 1, wherein the anchor cloth, the waist straps, the lower limb straps, and the thigh straps are made of a non-elastic, baby-friendly cloth.

6. The body restraint apparatus of claim 1, wherein the first angle is about 40° and the second angle is about 40° such that an angle of about 80° is formed between the lower limb straps.

7. The body restraint apparatus of claim 1, wherein the fastener portions are hook fastener portions and loop fastener portions.

8. The body restraint apparatus of claim 7, wherein the hook fastener portions are narrower than the loop fastener portions.

9. A method of restraining a person in a frog leg position comprising:
fastening a first waist strap to a second waist strap to form a waist restraint around a waist of the person;
fastening a first thigh strap to a second thigh strap to form a first thigh restraint around a first lower thigh of the person in the frog leg position; and
fastening a third thigh strap to a fourth thigh strap around a second lower thigh of the person in the frog leg position;
wherein the waist straps and thigh straps are coupled to an anchor cloth located behind a lower back of the person and are sized to restrain movement from the frog leg position.

10. The method of claim 9 further comprising:
fastening a first calf strap to a second calf strap to form a first calf restraint around a first upper calf of the person in the frog leg position; and
fastening a third calf strap to a fourth calf strap around a second upper calf of the person in the frog leg position;
wherein the calf straps are coupled to the anchor cloth and are sized to restrain movement from the frog leg position.

11. The method of claim 9 further comprising:
placing a shoulder roll under an upper back and shoulders of the person and a head support roll extending from the shoulder roll under a head of the person;
fastening a first wrist strap extending from the shoulder roll to form a first wrist restraint around a first wrist of the person in the frog leg position; and
fastening a second wrist strap extending from the shoulder roll to form a second wrist restraint around a second wrist of the person in the frog leg position.

12. The method of claim 9 further comprising:
fastening a first ankle strap to form a first ankle restraint around a first ankle of the person in the frog leg position; and
fastening a second ankle strap to form a second ankle restraint around a second ankle of the person in the frog leg position.

13. A CPAP restraint apparatus comprising:
an anchor cloth having a first side edge and a second side edge opposite the first side edge;
a first forehead strap extending sideward from a top portion of the first side edge of the anchor cloth;
a first forehead strap fastener portion located on a distal portion of the first forehead strap;
a second forehead strap extending sideward from a top portion of the second side edge of the anchor cloth;
a second forehead strap fastener portion, complementary to the first forehead strap fastener portion, located on a distal portion of the second forehead strap;
a first lip strap extending sideward from a bottom portion of the first side edge of the anchor cloth;
a first lip strap fastener portion located on a first portion of the first lip strap;
a second lip strap fastener portion, complementary to the first lip strap fastener portion, located on a second portion of the first lip strap;
a second lip strap extending sideward from a bottom portion of the second side edge of the anchor cloth;
a third lip strap fastener portion located on a first portion of the second lip strap;
a fourth lip strap fastener portion, complementary to the third lip strap fastener portion, located on a second portion of the second lip strap;
a first chin strap extending from a bottom edge of the first lip strap near the proximal end of the first lip strap;
a first chin strap fastener portion located on a distal portion of the first chin strap;
a second chin strap extending from a bottom edge of the second lip strap near the proximal end of the first second strap;
a second chin strap fastener portion located on a distal portion of the second chin strap;
wherein the anchor cloth, the forehead straps, the lip straps, and the chin straps are sized such that, with the anchor cloth located behind a head of a person using CPAP tubing, the first forehead strap fastener portion and the second forehead strap fastener portion fasten together to form a forehead restraint around a forehead of the person and a portion of the CPAP tubing, the first lip strap fastener portion and the second lip strap fastener portion fasten together to form a first tubing restraint around a first tubing elbow of the CPAP tubing, the third lip strap fastener portion and the fourth lip strap fastener portion fasten together to form a second tubing restraint around a second tubing elbow of the CPAP tubing, and the first chin strap fastener portion and the second first chin strap fastener portion fasten together to form a chin restraint under a chin of the person.

14. The CPAP restraint apparatus of claim 13, wherein the person is a neonate.

15. The CPAP restraint apparatus of claim 13, wherein the anchor cloth, the waist straps, the lower limb straps, and the thigh straps are made of a non-elastic, baby-friendly cloth.

16. The CPAP restraint apparatus of claim 13, wherein the fastener portions are hook fastener portions and loop fastener portions.

17. The CPAP restraint apparatus of claim 13, wherein the hook fastener portions are narrower than the loop fastener portions.

18. The CPAP restraint apparatus of claim 13 further comprising an orogastric tube holder attached to the second chin strap.

19. The CPAP restraint apparatus of claim 13, wherein the orogastric tube holder comprises a soft plastic rectangular strip and a strip of adhesive tape with a paper tab.

20. A method of restraining a person wearing a CPAP comprising:
fastening a first forehead strap to a second forehead strap to form a forehead restraint around a forehead of the person and CPAP tubing extending across the forehead;
fastening a first lip strap to form a first tubing restraint around a first CPAP tubing elbow;
fastening a second lip strap to form a second tubing restraint around a second CPAP tubing elbow; and
fastening a first chin strap to a second chin strap to form a chin restraint under a chin of the person;
wherein the forehead straps, lip straps, and chin straps are coupled to an anchor cloth located behind a head of the person and are sized to restrain movement of the chin and movement of the CPAP tubing with respect to the head of the person.

21. The method of claim 20 further comprising attaching an orogastric tube to an orogastric tube holder attached to the second chin strap.

22. The method of claim 20 further comprising:
fastening a first wrist strap to form a first wrist restraint around a first wrist of the person; and
fastening a second wrist strap to form a second wrist restraint around a second wrist of the person;
wherein the wrist straps are coupled to a stabilizer cloth located behind the back of the person and are sized to restrain an upper limb of the person from touching the head of the person, the forehead straps, the lip straps, the chin straps, the CPAP tubing or the orogastric tube.
